(19) United States
(12) Patent Application Publication
(10) Pub. No.: US 2022/0251545 A1
(43) Pub. Date: Aug. 11, 2022

(54) FRAGMENT-BASED SCREENING TO IDENTIFY SMALL MOLECULES THAT SELECTIVELY BIND RNA

(71) Applicant: THE SCRIPPS RESEARCH INSTITUTE, La Jolla, CA (US)

(72) Inventor: Matthew D. Disney, Jupiter, FL (US)

(21) Appl. No.: 17/622,032

(22) PCT Filed: Jun. 25, 2020
(86) PCT No.: PCT/US2020/070189

§ 371 Date: Dec. 22, 2021

Related U.S. Application Data
(60) Provisional application No. 62/867,607, filed on Jun. 27, 2019.

(51) Int. Cl. C12N 15/10; G01N 33/543

(52) U.S. Cl. CPC C12N 15/1065; G01N 33/54326

(57) ABSTRACT

A method is described to define the binding of fragments onto RNA targets and to use this profiling to enable the design of small molecules targeting RNA. The method comprises exposing a labeled RNA target to a small molecule fragment appended with diazirine and an alkyne moiety. Exposure of the compounds to light produce a reactive intermediate from the diazirine moiety that will react with sites in the RNA that are proximal to the small molecule fragments binding site. The RNAs that are reacted with the fragments are captured by using a biotin azide or azide-displaying beads that react with the alkyne moiety in the presence of a Cu(I) catalyst using click chemistry. Biotinylated products are captured with streptavidin resin. The amount of labeled RNA captured by the resin/beads is measured, thereby identifying which fragments bind an RNA target. The binding site of the fragment is determined by RT-PCR.

### CLAIM OF PRIORITY

The present application claims the benefit of priority to U.S. Provisional Application No. 62/867,607 filed on Jun. 27, 2019, which application is incorporated by reference as if fully set forth herein.

### STATEMENT OF GOVERNMENT SUPPORT

This invention was made with government support under grant number GM97455 awarded by the National Institutes of Health. The government has certain rights in the invention.

### BACKGROUND

The aberrant expression or mutation of RNAs cause or contribute to nearly every human disease and thus are critical targets for therapeutic intervention. The identification of small molecules that target RNA is challenging, however.

### SUMMARY

The overall structure of an RNA is comprised of composites of three dimensionally folded elements. One manner to discover compounds that bind a target RNA is by identifying small molecule fragments these three dimensionally folded elements. The fragments can then be linked together to afford compounds that are higher affinity and more selective than the individual fragments. The issue with deciphering the fragments that bind RNA and their binding sites is that many of them bind with low affinity and have short residence times. This challenge can be overcome by using fragments capable of covalent binding. In this approach, a fragment is appended with a group that can react with a target, forming a covalent bond and essentially freezing the complex. The covalent adduct can then be isolated and studied to decipher, for example, the extent and site of reaction. Footprinting reactive sites allows one to position individual fragments for binding to the desired target and then custom-linking the fragments to afford a multimeric compound that is a higher affinity binder relative to the fragments from which they are derived.

The invention provides, in various embodiments, a method to identify fragments that bind RNA that can be custom assembled to afford a multimeric compound that binds multiple sites in the RNA simultaneously, increasing affinity and specificity. That is, both small molecule fragments and their binding sites within the RNA are identified, allowing rational design of small molecules that target disease-causing RNAs. Knowledge of the set of RNA-binding fragments can be used to modularly assemble the plurality of fragments into a multimeric compound that binds the RNA target with high affinity and specificity.

As used herein, a “fragment” is an organic chemical structure or substructure, for which RNA binding affinity is tested. The “fragment” or “substructure” of the present invention comprises a potential RNA-binding moiety (“fragment”) having a linker group, such as a carboxylic acid group, that can be coupled with an alkyne-containing moiety for Click Chemistry reaction with an azide, and a diazirene-containing moiety. The assembled structure comprising the RNA-binding moiety, the alkyne-containing moiety, and a diazirene-containing moiety, is referred to herein as a “substructure reagent”.

### BRIEF DESCRIPTION OF THE FIGURES

FIG. 1 depicts a schematic illustration of steps of practicing a method of the invention.

FIG. 2 is a bar graph illustrating the binding of test compound TGP-21-diaz comprising test RNA-binding substructure covalently linked with alkyne and diazirene groups, with pre-miR-21 RNA (radiolabeled). As a control, the non-binding but photoreactive compound “methyl-diaz”, a small molecule comprising the alkyne and the diazirene groups but lacking the test RNA-binding substructure is used.

FIG. 3 is a bar graph illustrating the binding of test compound TGP-21-diaz comprising test RNA-binding substructure covalently linked with alkyne and diazirene groups, with pre-miR-21 RNA (radiolabeled) can be competed off with small molecule fragments to identify new RNA-binding substructures that lack a diazirine cross-linking module.

### DETAILED DESCRIPTION

We have developed a method to map the binding sites of small molecules targeting RNA by appending them with a diazirine cross linker and an alkyne handle to install a purification module, i.e., biotin azide, or to conjugate directly to azide-displaying beads. The approach, dubbed Chem-CLIP-Map, is enabled by initiating a photochemically-induced transformation of diazirine into a reactive species that will react with sites in an RNA target that are close in space. This reaction installs a covalent adduct onto the RNA target that also adds a biorthogonal reactive group suitable for purifying away RNAs that have been cross-linked vs. RNAs that have not. The RNAs that reacted with the small molecule are identified by purification of the cellular lysate by a Huisgen dipolar cycloaddition reaction between the alkyne handle and (i) biotin-azide following by incubation with streptavidin resin; or (ii) azide-displaying beads. The resin is washed, and the bound material is isolated by eluting bound adducts. The linker between the biotin group and the streptavidin or fragment and the bead comprises a disulfide linkage that can be cleaved reductively.

The fragment binding site of the RNA in the adduct is detected by subjecting the RNA to reverse transcription to produce a cDNA that is then PCR amplified. Reverse transcriptase is unable to read through the cross-link, and therefore the reverse transcription reaction and hence PCR amplification is terminated at the site of reaction. The amplified fragments can then be mapped onto the sequence of the intact RNA to identify the binding sites on the RNA of the fragments tested.

In the present invention, libraries of small molecule fragments are screened for binding to RNA. The fragments are appended to a diazirine reactive module and an alkynyl tag to allow for installation of purification tags after reaction. To initiate an experiment, a labeled RNA, whether with a fluorescent or radioactive tag, is incubated with a diazirine library individually. Upon irradiation with light, the fragments that are bound to the RNA form a covalent cross-link with the target. The reaction is then captured onto streptavidin resin and the washed. The amount of labeled RNA is then quantified.

Thus, in various embodiments, the invention provides a method of identifying an RNA-binding substructure in a library comprising a plurality of substructures, comprising

preparing a library comprising members, wherein each member comprises one of the plurality of substructures in form of a respective substructure reagent, wherein each substructure reagent comprises the respective substructure covalently conjugated to a moiety comprising an alkyne group and to a moiety comprising a diazirene group; then,

contacting the library comprising the plurality of substructure reagents, and the RNA target for which the RNA-binding substructure is sought, wherein the RNA comprises a radioactive label or a fluorescent label to provide an RNA-contacted library; then,

illuminating the RNA-contacted library with UV light under conditions to cause conversion of a diazirene group to a reactive carbene, which carbene reacts covalently with the RNA with which any RNA-binding substructure of the library of substructure reagents is associated non-covalently; then,

contacting the library of substructures and associated radiolabeled RNA, and a pull-down reagent comprising a biotin group and an azide group, under conditions such that the azide group of the pull-down reagent and the alkyne group of the substructure reagent react to form a triazole ring, providing a biotinylated substructure reagent, covalently bound to RNA only when the RNA-binding substructure is present in that member of the library; then,

contacting each of the members of the library with a respective streptavidin magnetic bead, pulling down each of the biotinylated substructure reagents, then separating the beads from supernatant liquid magnetically;

or contacting the library of substructures and associated radiolabeled RNA, and a pull-down reagent comprising beads with an azide group, under conditions such that the azide group of the pull-down reagent and the alkyne group of the substructure reagent react to form a triazole ring, providing a biotinylated substructure reagent, covalently bound to RNA only when the RNA-binding substructure is present in that member of the library;

then

measuring radioactivity or fluorescence associated with each member of the library, wherein each substructure reagent covalently associated with RNA that is bound to a bead is identified as an RNA-binding substructure.

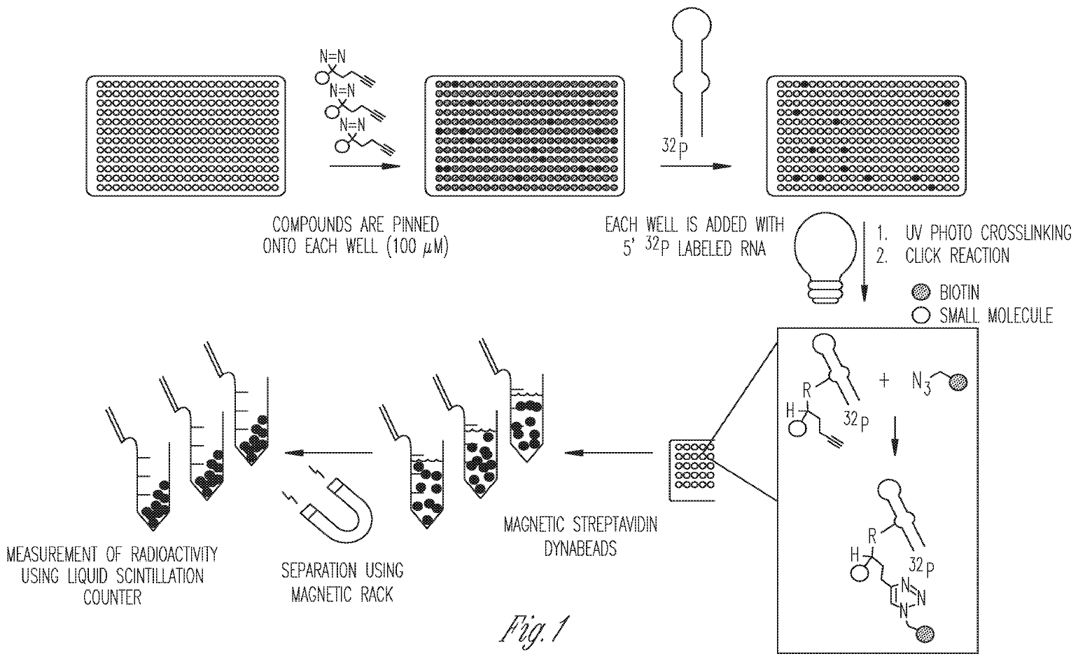

FIG. 1 provides a schematic outline of practice of a method of the invention. A segment of RNA which is to be probed for binding of one or more members of a molecular library is prepared with a ³²P-radiolabel or with a fluorescent label. An appropriate format for a multi-member library, such as a multi-well plate, e.g. a 384-well plate, is loaded (“pinned”) with a plurality of substructure reagents, where each substructure reagent comprises the respective substructure covalently conjugated to a moiety comprising an alkyne group and to a moiety comprising a diazirene group, being pinned in one or more of the test wells while other test substructures are pinned in other wells. A substructure can be any molecular entity desired for evaluation of its affinity for an RNA target. The substructure to be tested comprises a site for linkage of the moiety comprising an alkyne group and the moiety comprising a diazirene group. For example, the site for linkage of the substructure to be tested to form the substructure reagent, to be for pinning in one or more test wells of the multi-well plate, can be a carboxylic acid group.

An example is found in the substructure reagent TGP-21-diaz, which was evaluated according to the method of the invention for binding to pre-miR-21. The test substructure TGP-21-Acid was coupled with the alkyne-containing moiety prepared as shown in Synthetic Scheme 1, then with the diazirene-containing moiety “diazirine acid” (see Synthetic Scheme 2), to obtain the substructure reagent TGP-21-diaz.

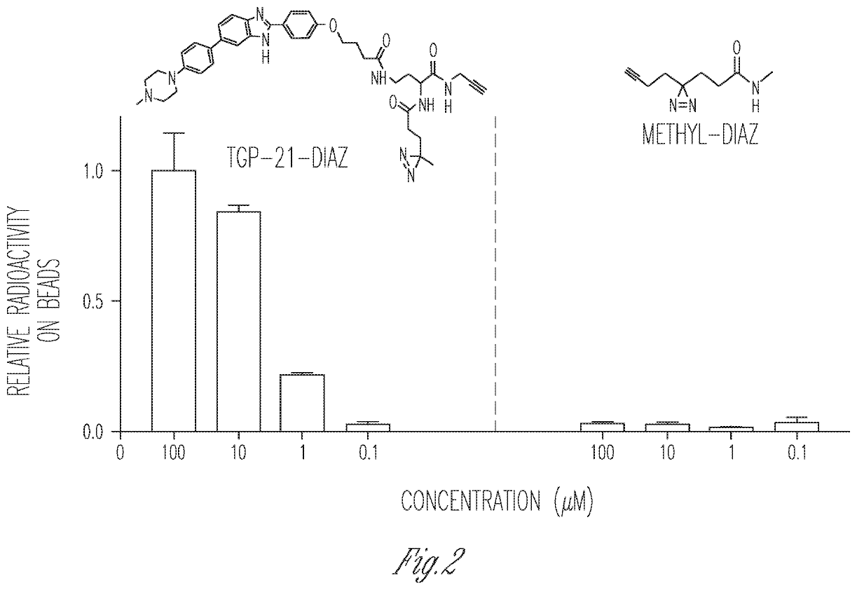

FIG. 2 shows the bioactivity of the TGP-21-diaz in binding the RNA pre-miR-21 at various concentrations, with the compound “methyl-diaz” being a non-binding but alkyne- and diazirene-containing control molecule. A concentration of about 10 μM TGP-21-diaz is sufficient to bring about covalent binding with the pre-miR-21 RNA (as shown by incorporation of the RNA radiolabel onto the magnetic streptavidin beads according to the method of the invention). Accordingly, TGP-21-diaz can be identified in the presence of a plurality of other substructure reagents as having affinity for the pre-miR-21 RNA. In this manner, other potential RNA-binding substructures can be identified.

As shown in FIG. 1, following the pinning of the plurality of substructure reagents each representing a substructure to be tested for binding the target RNA, the target RNA bearing either a radioactive or a fluorescent reporter label is introduced into each of the wells, followed by illumination with UV light sufficient to bring about decomposition of the diazirene moiety to produce a carbene group, which due to its high reactivity with covalent bonds, inserts into a bond of the RNA molecule with which the substructure is non-covalently associated. When there is not binding of the substructure to the RNA, the reactive carbene is not in proximity to any RNA, so no covalent bond is formed between carbene and RNA.

Then, the alkyne-containing moiety, now covalently linked to RNA for those library members where there is substructure-RNA binding, but not for those library members where there is not RNA binding, is reacted using the copper-catalyzed “click chemistry” of alkyne and azide to append a biotin-containing group or to azide-displaying beads. FIG. 1 shows the resulting structures graphically in the box following step 2, “click reaction”.

The appended biotin group allows pull-down of the substructure reagent now bound to the magnetic streptavidin bead, with or without covalently linked labeled RNA as an indicator of binding. Separation of the beads, and assay of the RNA reporter group (radioactive atom or fluorescent group) bound thereto, indicates those substructures having affinity for the target RNA.

As an illustration of the principles of the screening method of the invention, an RNA-binding substructure, TGP-21-acid, was tested for its binding of the RNA “pre-miR-21”. Synthetic Scheme 1, below, outlines the steps used for conversion of the RNA-binding substructure TGP-21-acid to the photoaffinity labeling product TGP-21-diaz. The RNA-binding substructure comprising the N-methylpiperazinyl-phenyl-benzimidazole-phenyl moiety is derivatized to provide a reactive carboxylic acid group as a butyric acid chain bonded via an ether linkage of the terminal carbon atom to the aromatic RNA-binding substructure, providing TGP-21-acid.

This substructure reagent incorporating both the putative RNA-binding group and the carboxylic acid functional handle can be converted by the reaction sequence shown to provide the final reagent for testing. First, the carboxylic acid group is converted to an amide with the alkyne-containing reagent, compound 2 of Synthetic Scheme 2, shown in step 1 of Synthetic Scheme 1. This reagent can be prepared according to Synthetic Scheme 2, below. The α-Fmoc 2,4-diaminobutyrate propargylamide compound 2 provides both the alkynyl group for subsequent click chemistry reaction with an azide, and an amino group that can be further derivatized in the diazirene acid shown in step 3 of Synthetic Scheme 1.

Similarly, other potential RNA-binding substructures can be investigated for determining their RNA target in an analogous manner, using the analogous substructure (fragment) reagent bearing both the alkyne group for subsequent click chemistry coupling with an azide reagent and the diazirene group for photoactivated covalent coupling to the RNA molecule to which the substructure reagent is bound non-covalently. A library of potential RNA-binding substructures (fragments) can be investigated for binding for any target RNA of interest by converting each respective substructure to a substructure reagent in which the substructure to be tested is covalently conjugated via a carboxylic acid group to a moiety comprising an alkyne group and to a moiety comprising a diazirene group. Thus, a substructure reagent such as TGP-21-diaz can be prepared for each of a plurality of substructures other than the N-methylpiperazinyl-phenyl-benzimidazole-phenyl moiety tested in TGP-21-diaz.

A library of test substructures, each loaded (“pinned”) in a well of a multi-well a multi-member library, such as a mufti-well plate, is exposed to the target RNA which has been labeled with a detectable label such as a radioactive atom (e.g., ³²P) or a fluorescent reporter group. After non-covalent binding of the RNA occurs to those library members having an affinity for that target RNA, light activation of the photoreactive diazirene group causes a covalent bond to form between the substructure reagent and the labeled RNA. The covalent bond prevents dissociation of the substructure reagent and the labeled RNA in subsequent processing steps.

Then, a copper-catalyzed alkyne-azide click chemistry reaction of the covalently RNA-bound substructure reagent comprising an alkyne group, and a biotin-containing “pull-down” reagent comprising an azide group, provides for those wells where a covalent RNA-substructure reagent complex exists (i.e., where RNA binding of the substructure reagent occurred), a biotinylated substructure reagent covalently bound to the target RNA.

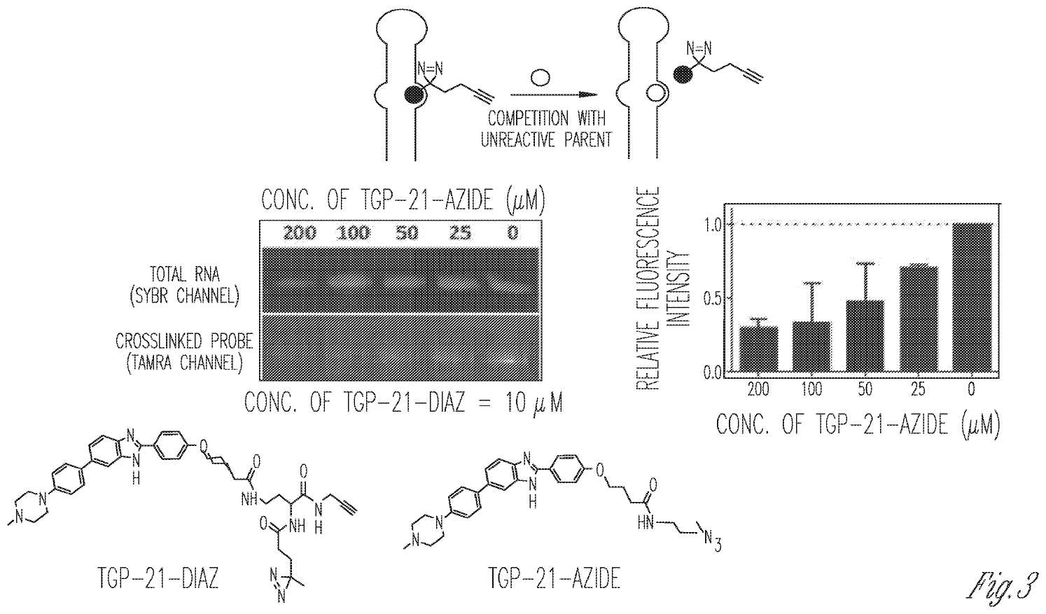

Next, magnetic beads comprising a streptavidin affinity reagent for the biotin group are introduced into each of the test wells of the multi-well library. Those wells in which a covalent complex of the substructure reagent and the labeled RNA exist, after the introduction of the biotin-containing pull-down reagent, are identified by detection of the RNA label, radioactive or fluorescent, in association with the magnetic beads. Those wells in which the RNA label is detected on the pulled-down beads allow identification of which substructures (fragments) formed a bound association with the target RNA. In this manner, one or more RNA-binding substructures of an RNA target can be identified from among a library of potential RNA-binding substructures (FIG. 3).

Alternatively, a copper-catalyzed alkyne-azide click chemistry reaction of the covalently RNA-bound substructure reagent comprising an alkyne group, and azide-displaying magnetic beads, provides for those wells where a covalent RNA-substructure reagent complex exists (i.e., where RNA binding of the substructure reagent occurred), beads covalently bound to the target RNA.

Those wells in which a covalent complex of the substructure reagent and the labeled RNA exist, after the introduction of the azide-displaying beads, are identified by detection of the RNA label, radioactive or fluorescent, in association with the magnetic beads. Those wells in which the RNA label is detected on the pulled-down beads allow identification of which substructures (fragments) formed a bound association with the target RNA. In this manner, one or more RNA-binding substructures of an RNA target can be identified from among a library of potential RNA-binding substructures.

### Examples

### Synthetic Scheme 1: Preparation of TGP-21-Diaz Substructure Reagent

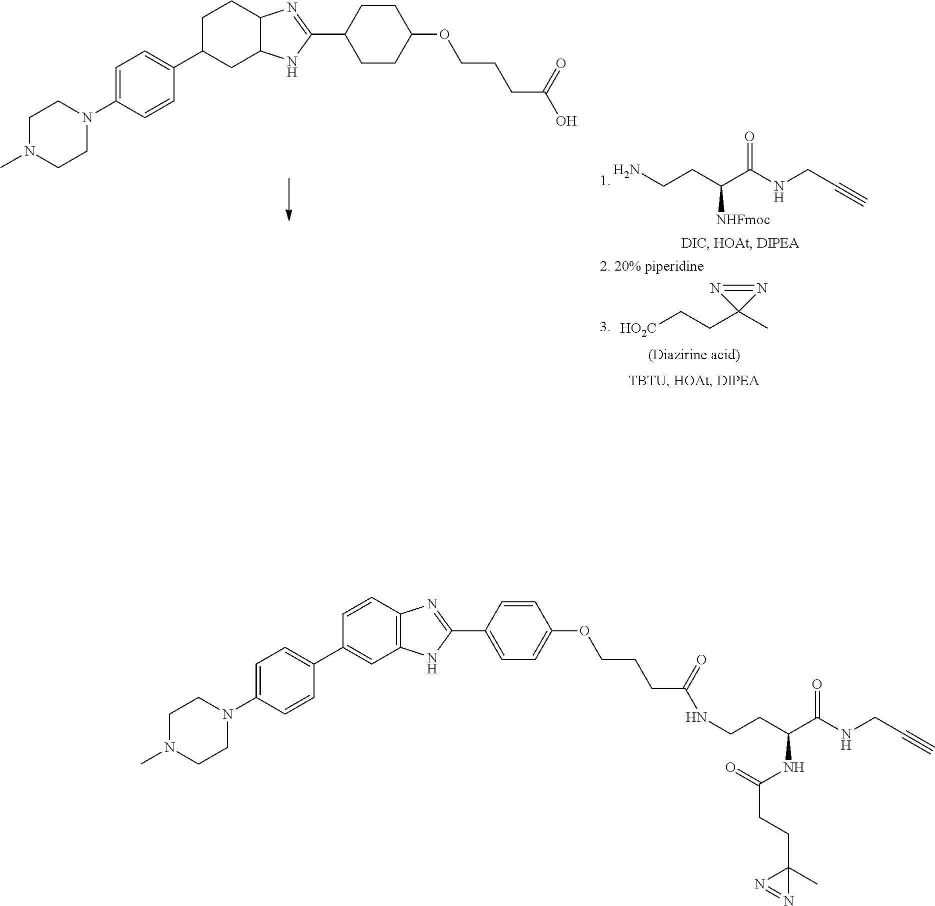

### Methods:

Synthesis of 1: Fmoc-Dab(Boc)-OH (CAS: 125238-99-5, 1 g, 2.27 mmol) was taken in a round bottom flask and was dissolved in 200 mL of dichloromethane (DCM). To this solution was added N,N-diisopropylcarbodiimide (DIC) (430 μL, 2.72 mmol). After stirring the reaction mixture at room temperature for 15 min, propargylamine (235 μL, 3.41 mmol) was added dropwise. The reaction was stirred at room temperature overnight, forming a white precipitate. After confirming the reaction went to completion by thin layer chromatography (TLC) and on reaction completion, the precipitate was filtered, washed with water, and dried. The white powder obtained was then purified by flash chromatography to obtain the desired product 1 (0.76 g; 70% yield).

Synthesis of 2: Compound 1 (700 mg) was placed in a round bottom flask with 50 mL of 30% trifluoracetic acid (TFA) in DCM. The solution was then stirred at room temperature for 30 min. Reaction progress was monitored by TLC and upon completion, the solvent was evaporated. The residue obtained was then dissolved in DCM and purified by flash chromatography to obtain desired product 2 (420 mg; 76% yield).

Synthesis of TGP-21-diaz. TGP-21-acid (5 mg, 10.63 μmol) was synthesized as previously described¹ and dissolved in 250 μL of N,N-dimethylformamide (DMF). DIC (1.7 μL, 10.63 μmol), 1-hydroxy-7-azabenzotriazole (HOAt; 1.45 mg, 10.63 μmol), and N,N-diisopropylethylamine (DIPEA: 2 μL, 10.63 μmol) were then added. After stirring the reaction mixture at room temperature for 15 min, compound 2 (4.01 mg, 10.63 μmol) dissolved in 50 μL of DMF was added to the reaction mixture and stirred at room temperature. Reaction progress was monitored via MALDI-TOF mass spectrometry (MS). Upon reaction completion, the solvent was evaporated, and the resulting residue was purified by high performance liquid chromatography (HPLC; methanol:water—0 to 100% over 60 min). The product's mass was confirmed via MALDI-TOF MS. See Synthetic Scheme 1, below.

The compound was dissolved in DMF containing 20% piperidine (200 μL), and the reaction was stirred at room temperature for 30 min. After confirming product formation by MALDI-TOF MS, the solvent was evaporated. The residue was dissolved in 100 μL of followed by addition of 100 mL of DMF containing 2-(1H-Benzotriazole-1-yl)-1,1,3,3-tetramethylaminium tetrafluoroborate (TBTU; 792 μg, 2.47 μmol), 1-Hydroxy-7-azabenzotriazole (HOAt, 448 μg, 3.29 μmol), DIPEA (0.6 μL, 3.29 μmol) and diazirine acid (0.6 μg, 4.9 μmol; see synthetic procedure below). The reaction was stirred at room temperature overnight. Product formation was confirmed by MALDI-TOF MS, after which the solvent was removed under vacuum. The residue was purified by HPLC (methanol:water—0 to 100% over 60 min). The mass of desired product, TGP-21-diaz was confirmed by MALDI-TOF MS.

### Synthesis of Diazirine Acid

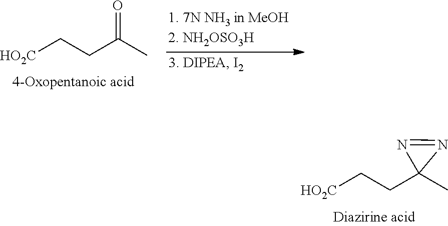

A round bottom flask containing 4-oxopentanoic acid (1 g, 8.61 mmol) was cooled to 0° C. and was charged with 7 N NH₃ in methanol (100 mL). The resulting solution was stirred at 0° C. under argon for 3 h. After, a solution of hydroxylamine-O-sulfonic acid (1.17 g, 10.33 mmol) in anhydrous methanol (10 mL) was added dropwise via addition funnel at 0° C. The resulting solution was stirred at 0° C. for an additional 1 h and then allowed to warm to room temperature overnight. The resulting suspension was evaporated to dryness and resuspended in methanol (10 mL). The solid was filtered away and washed several times with methanol. The combined filtrates were evaporated and resuspended in anhydrous methanol (100 mL), then cooled to 0° C. (protected from light). Diisopropylethylamine (3.2 mL) was added, followed by iodine (portion-wise), until a dark brown color persisted for more than 30 min, indicating total oxidation of diaziridine. The solution was then diluted with ethyl acetate (100 mL) and washed with aq. 1 N HCl (100 mL), saturated aqueous sodium thiosulfate (3×, 100 mL) and brine (100 mL). The combined aqueous phases were washed once with ethyl acetate. All organic layers were combined, dried over anhydrous sodium sulfate, and solvent was removed under reduced pressure. The obtained crude product was purified by flash column chromatography to obtain desired product as light brown colored oil (105 mg, 9.5%).

### Synthetic Scheme 2: Synthesis of Diazirine-Containing Probe for Pre-miR-21

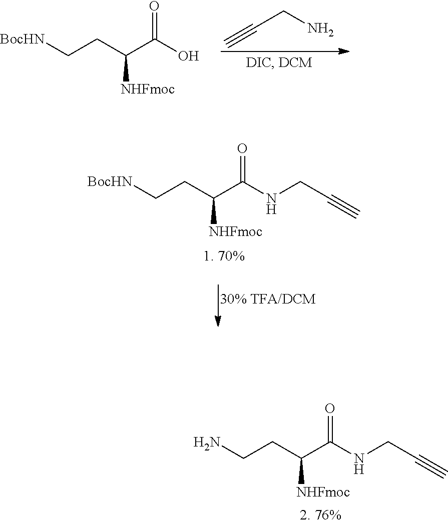

### In Vitro Methods:

Confirming reaction of TGP-21-diaz, a small molecule equipped with a diazirine cross-linking module. In vitro transcribed pre-miR-21 was dephosphorylated with calf intestinal alkaline phosphatase [CIAP; New England Biolabs (NEB)], radiolabeled with [³²P-γ]ATP and T4 polynucleotide kinase (NEB), and purified by denaturing gel electrophoresis as previously described.² The RNA (˜2000 cpm) was folded in 10 μL of 20 mM Hepes buffer, pH 7.5, by heating to 60° C. for 5 min followed by slowly cooling to room temperature. The RNA solution was then treated with TGP-21-diaz Chem-CLIP probe for 30 min at room temperature at varying concentrations to afford a dose response [0.1-100 μM]. Methyl-diaz was used as a negative control (0.1-100 μM); Methyl-diaz was incapable of pulling down pre-miR-21:

The samples were irradiated with UV light (365 nm) in a UVP Crosslinker (Analytik Jena) for 10 min. To each reaction was then added a “click mixture” [Biotin-PEG3-Azide (0.8 μL, 10 mM; Click Chemistry Tools), CuSO₄ (0.5 μL, 10 mM), Tris(3-hydroxypropyltriazolylmethyl)amine (THPTA; 0.1 μL, 50 mM) and sodium ascorbate (0.1 μL, 250 mM)]. The samples were incubated at 37° C. for 2 h followed by addition of 8 μL streptavidin beads (Dynabeads MyOne Streptavidin C1 beads; Thermo Scientific) to pull down crosslinked pre-miR-21. Un-reacted RNA, present in the supernatant, was removed by placing reaction tubes on a magnetic separation rack. The beads were washed twice with 1× Wash Buffer (10 mM Tris-HCl pH 7.0, 1 mM EDTA, 4 M NaCl, and 0.2% Tween). The radioactive signal associated with the beads and in the supernatant was measured by liquid scintillation counting.

Confirming Reaction of TGP-21-Diaz with Pre-miR-21: Fluorescence Assay.

To confirm the results cross-linking studies with radioactively labeled pri-miR-21, in vitro transcribed pri-miR-21 RNA (5 μM in total volume of 10 μL) was treated with TGP-21-diaz at various concentrations (12.5-100 μM) for 30 min at room temperature. For competition experiment, pre-miR-21 was first incubated with 10 μM of TGP-21-diaz for 15 min at room temperature followed by various concentration of the parent, unreactive compound TGP-21 (25-200 μM). The reaction was then irradiated with UV light (365 nm) using UVP Crosslinker for 10 min. After irradiation, the samples were supplemented with a click reaction mixture containing tetramethylrhodamine (TAMRA) azide (0.5 μL, 100 mM; Click Chemistry Tools), CuSO₄ (1 μL, 10 mM), THPTA (1 μL, 50 mM) and sodium ascorbate (1 μL, 250 mM). The click reaction was incubated at 37° C. for 2 h. The RNA was ethanol precipitated, which also removed unreacted components of the click reaction mixture. The RNA was dissolved in water and subjected to denaturing polyacrylamide gel electrophoresis. The gel was image using a Molecular Dynamics Typhoon 9000. First, fluorimaging was completed in the TAMRA channel (Ex: 546 nm; Em: 579 nm) to visualize cross-linked products. The gel was then stained with SYBR green and imaged to visualize all RNA (Ex: 497 nm; Em: 520 nm).

Identification of compound binding sites via primer extension or sequencing. In vitro transcribed pri-miR-21 (100 μL of 5 μM) was folded in 20 mM Hepes, pH 7.5, as described above and treated with varying concentrations of TGP-21-diaz (1, 10, and 100 μM) for 30 min at room temperature. The samples were then irradiated with UV light (365 nm) using a UVP Crosslinker for 10 min. Akin to the studies described above, the reaction was then supplemented with a click reaction mixture, in this case consisting of disulfide biotin azide (0.5 μL, 100 mM; Click Chemistry Tools), CuSO₄ (1 μL, 10 mM), THPTA (1 μL, 50 mM) and sodium ascorbate (1 μL, 250 mM). The reaction was incubated at 37° C. for 2 h, and the samples were ethanol precipitated. The RNA was reconstituted in 10 μL of Nanopure water and incubated with 10 μL of streptavidin beads (Dynabeads MyOne Streptavidin C1 beads; Thermo Scientific). After a 30 min incubation at room temperature, un-reacted RNA was removed by placing reaction tubes on a magnetic separation rack. The beads were washed twice with 1× Wash Buffer.

The cross-linked RNA associated with the beads was eluted by reducing the disulfide bond present in the biotin used in the click reaction mixture. In brief, the beads were incubated with 50 μL of a prepared mixture containing 1:1 200 mM tris(2-carboxyethyl)phosphine (TCEP) and 600 mM K₂CO₃ for 30 min at 37° C. The thiols were then capped with iodoacetamide (50 μL, 400 mM) for 30 min at room temperature (protected from light). The beads were removed using a magnetic rack, and the eluted RNA in the supernatant was transferred to a new tube and ethanol precipitated.

Compound binding sites can be identified by reverse transcription (RT). That is, RT is unable to proceed through nucleotides where the compound has been cross-linked. For these ligation-mediated PCR, or Chem-CLIP-Map-Seq, a gene-specific RT primer was used (5′-³²P-CAGACGTGCTCTTCCGATCTCCTTGCGTCAGACAGCC), SEQ ID NO:1. After the RT step, the RNA was digested with 0.5 μL RNase H and 0.5 μL RNase A for 30 min at 37° C. The RT reaction was then cleaned up using Agencourt RNAClean XP beads per manufacturer's protocol for small RNAs. The purified RT reaction was ligated to a ssDNA (5′Phos-NNNAGATCGGAAGAGCGTCGTGTAG-3Cspacer), SEQ ID NO:2, using T4 RNA ligase I (NEB). Briefly, 2 μL 10×T4 RNA ligase buffer, 1 μL 1 mM ATP, 10 μL 50% PEG 8000, 5 μL cDNA, 1 μL of 20 μM ssDNA adaptor, and 1 μL of T4 RNA ligase were incubated overnight at RT.³ This reaction mixture was cleaned up using RNA Clean XP as described above for RT reactions. The ligated cDNA was amplified using the following primers and Phusion polymerase (NEB): 5′-CTACACGACGCTCTTCCGATCT-3′ and 5′-CAGACGTGCTCTTCCGAT-3′, SEQ ID NO:3. The PCR products and primers were separated on a denaturing 12% polyacrylamide gel containing SYBR green. The desired band was excised, and the DNA was isolated by tumbling the gel overnight in 0.3 M sodium chloride solution followed by ethanol precipitation. The isolated cDNA was cloned into NEB's pminiT 2.0 vector per manufacturer's protocol and sequenced by Eton Biosciences.

Screening of small molecule fragments containing diazirines. A 5 μL aliquot of 20 mM Hepes buffer, pH 7.5, was added to each well of a 384-well plate (white; Greiner catalog #784065). Next, 100 nL of compound (10 mM) was transferred to the wells with a Beckman Coulter Biomek pintool. In vitro transcribed, 5′-³²P labeled pre-miR-21 (1 μL; ˜400000 cpm) was folded in 1300 μL of 20 mM Hepes, pH 7.5, by heating at 60° C. for 5 min followed by slowly cooling to room temperature. A 5 μL aliquot of the folded RNA was then added to each well of the 384-well screening plate, and the samples were incubated at room temperature for 30 min. The samples were then irradiated with UV light (365 nm) for 10 min with Stratalinker UV Crosslinker. After irradiation, a click mixture (PEG3 biotin azide (0.8 μL, 10 mM), CuSO₄ (0.5 μL, 10 mM), THPTA (0.1 μL, 50 mM) and sodium ascorbate (0.1 μL, 250 mM) was added to each well, and the samples were incubated at 37° C. for 2 h. Next, 8 μL of streptavidin magnetic beads (Dynabeads MyOne Streptavidin C1 beads; Thermo Scientific) were added to each well, and the samples were transferred to a 48-well plate. Unreacted RNA in the supernatant was removed from the beads using a 96-well magnetic separation rack. The beads were washed twice with 1× Wash Buffer. Radioactive signal associated with the beads and in the supernatant was measured by liquid scintillation counting.

### DOCUMENTS CITED

[1] Velagapudi, S. P., Luo, Y., Tran, T., Haniff, H. S., Nakai, Y., Fallahi, M., Martinez, G. J., Childs-Disney, J. L., and Disney, M. D. (2017) Defining RNA-small molecule affinity landscapes enables design of a small molecule inhibitor of an oncogenic noncoding RNA. ACS Cent Sci3, 205-216.
[2] Velagapudi, S. P., Gallo, S. M., and Disney, M. D. (2014) Sequence-based design of bioactive small molecules that target precursor microRNAs. Nat Chem Biol 10, 291-297.
[3] Sexton, A. N., Wang, P. Y., Rutenberg-Schoenberg, M., and Simon, M. D. (2017) Interpreting reverse transcriptase termination and mutation events for greater insight into the chemical probing of RNA. Biochemistry 56, 4713-4721.

What is claimed is:

1. A method of identifying an RNA-binding substructure in a library comprising a plurality of substructures, comprising
preparing a library comprising members, wherein each member comprises one of the plurality of substructures in form of a respective substructure reagent, wherein each substructure reagent comprises the respective substructure covalently conjugated to a moiety comprising an alkyne group and to a moiety comprising a diazirene group; then,
contacting the library comprising the plurality of substructure reagents, and the RNA target for which the RNA-binding substructure is sought, wherein the RNA comprises a radioactive label or a fluorescent label, to provide an RNA-contacted library; then,
illuminating the RNA-contacted library with UV light under conditions to cause conversion of a diazirene group to a reactive carbene, which carbene reacts covalently with the RNA with which any RNA-binding substructure of the library of substructure reagents is associated non-covalently; then,
contacting the library of substructures and associated radiolabeled RNA an azide-bearing group further comprising a pull-down group comprising a biotin moiety linked to the azide by a linker group comprising a disulfide bond, or alternatively with a bead bearing an azide group bonded thereto by a linker comprising a disulfide bond, under conditions comprising the presence of copper catalyst to bring about a CuAAC reaction, providing a biotinylated substructure reagent or bead, covalently bound to RNA only when the RNA-binding substructure is present in that member of the library; then,
contacting biotinylated substructure reagent each of the members of the library with a respective streptavidin magnetic bead, pulling down each of the biotinylated substructure reagents; then
separating the beads from supernatant liquid magnetically; then
measuring radioactivity or fluorescence associated with each member of the library, wherein each substructure reagent covalently associated with RNA that is bound to a magnetic bead is identified as an RNA-binding substructure; then
cleaving the SMIRNA-RNA covalent complex from the bead by reduction of the disulfide bond in the respective linker, to free the complex from the bead; then,
subjecting the RNA thus freed to reverse transcription and sequencing of the DNA from the reverse transcription process, to indicate the SMIRNA binding site on the RNA through the presence of a transcription stop.

2. The method of claim 1, wherein the substructure reagent is TGP-21-diaz

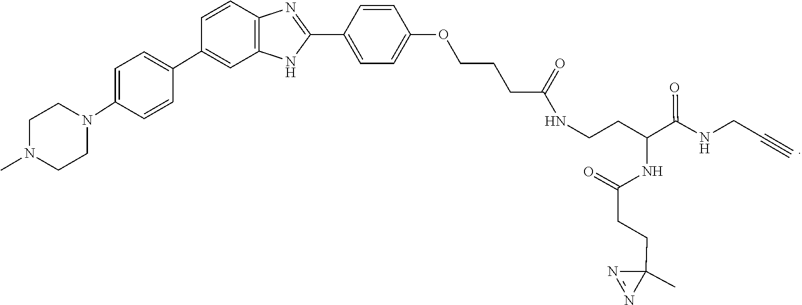 [SMILES: C#CCNC(=O)C(CCNC(=O)CCCOc1ccc(-c2nc3ccc(-c4ccc(N5CCN(C)CC5)cc4)cc3[nH]2)cc1)NC(=O)CCC1(C)N=N1]

3. The method of claim 1, wherein the RNA target is pre-miR-21.

4. The method of claim 1, wherein the moiety comprising an alkyne group is of formula

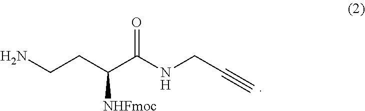

5. The method of claim 1, wherein the moiety comprising a diazirene group is of formula

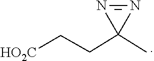

6. The method of claim 1, wherein the radioactive label of the target RNA comprises ³²P.

7. The method of claim 1, wherein the target RNA comprises a fluorescent label.

8. The method of claim 1, wherein the RNA comprises one or more of synthetic, semi-synthetic, or natural RNA.

9. The method of claim 1, wherein the RNA comprises the genome of an RNA virus.

10. The method of claim 1 carried out in vitro.

11. The method of claim 1 carried out in living cells.

12. The method of claim 1, wherein the living cells are virally- or bacterially-infected cells.

13. The method of claim 1 carried out in preclinical animal models.

14. The method of claim 1, wherein the method is performed in a parallel array for a plurality of RNA sequences and candidate RNA-binding small modules.
